(19) United States
(12) Patent Application Publication
(10) Pub. No.: US 2019/0274981 A1
(43) Pub. Date: Sep. 12, 2019

(54) OLEANOLIC ACID ENHANCES MESENCHYMAL STROMAL CELL OSTEOGENIC POTENTIAL BY INHIBITION OF NOTCH SIGNALING

(71) Applicant: Board of Supervisors of Louisiana State University and Agricultural and Mechanical College, Baton Rouge, LA (US)

(72) Inventor: Yufeng DONG, Shreveport, LA (US)

(21) Appl. No.: 16/264,664

(22) Filed: Jan. 31, 2019

Related U.S. Application Data
(60) Provisional application No. 62/625,286, filed on Feb. 1, 2018.

(51) Int. Cl. A61K 31/19; A61K 38/18; A61K 9/00

(52) U.S. Cl. CPC A61K 31/19; A61K 9/0019; A61K 38/1875

(57) ABSTRACT

A method of increasing mesenchymal stromal cell osteogenic potential in a patient comprising administering to the patient a therapeutic that inhibits Notch signaling. A method of stimulating one of bone tissue regeneration and bone tissue formation in a mammal comprising administering to the mammal a first therapeutic containing Oleanolic acid. A pharmaceutical product comprising a first therapeutic containing Oleanolic acid or a pharmaceutically acceptable salt, solvate, ester, amide, clathrate, stereoisomer, enantiomer, prodrug or analog thereof.

### CROSS REFERENCE TO RELATED APPLICATIONS/PRIORITY

The present invention claims priority to U.S. Provisional Patent Application No. 62/625,286 filed Feb. 1, 2018, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

Bone fracture healing and large bone defect healing are a large societal problem that is getting worse due to the increase of patients with bone loss caused by, for example, car accident, battlefield injury, and tumor resection. To date, surgery remains the best choice of treatment. More than one million traumatic fractures require surgical intervention in the US each year and approximately 10% result in delayed union or non-union due to reduced local mesenchymal stromal cell (MSC) osteogenic differentiation. Particularly in old people or in patients with diabetes, fractures take a long time to heal or may never heal, leading to long-term disability and/or amputation. Although growth factor bone morphogenetic protein 2 (BMP2) has been clinically used to enhance MSC differentiation toward bone forming cells during spine fusion surgery, adverse effects still remain, including osteoclast activation, life-threatening inflammatory swelling, and adipogenesis, when high dose of BMP2 is used. It is imperative to develop alternative bioactive agents to replace BMP2 or to be used in combination with BMP2, so as to reduce the dosage and the cost of BMP2. For the foregoing reasons, there is a pressing, but seemingly irresolvable need for developing an alternative treatment to enhance MSC differentiation toward bone forming cells.

### SUMMARY

Wherefore, it is an object of the present invention to overcome the above mentioned shortcomings and drawbacks associated with the current technology.

The present invention relates to pharmaceutical compositions of a therapeutic (e.g., oleanolic acid), or a pharmaceutically acceptable salt, solvate, ester, amide, clathrate, stereoisomer, enantiomer, prodrug or analogs thereof, and use of these compositions for the treatment of a condition requiring bone tissue regeneration or bone tissue formation, including bone fracture healing and large bone defect healing

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In some embodiments, the condition is a condition requiring bone tissue regeneration or bone tissue formation.

In certain embodiments, the condition requiring bone tissue regeneration or bone tissue formation is mild to moderate condition requiring bone tissue regeneration or bone tissue formation.

In further embodiments, the condition requiring bone tissue regeneration or bone tissue formation is moderate to severe condition requiring bone tissue regeneration or bone tissue formation.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more additional therapeutic agents for the treatment or prevention of the condition requiring bone tissue regeneration or bone tissue formation.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

As used herein, the term “delayed release” includes a pharmaceutical preparation, e.g., an orally administered formulation, which passes through the stomach substantially intact and dissolves in the small and/or large intestine (e.g., the colon). In some embodiments, delayed release of the active agent (e.g., a therapeutic as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably include a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” include pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coateric®), hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “immediate release” includes where the agent (e.g., therapeutic), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%, 65%, 75%, 85%, 90%, or 95% of the agent is released within the first 30 minutes, 45 minutes, or 60 minutes of administration.

The term “pharmaceutical composition,” as used herein, includes a composition containing a compound described herein (e.g., oleanolic acid, or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal.

Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, includes those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, oleate, oxalate, palm itate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, includes a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, includes prophylactic treatment or treatment that prevents one or more symptoms or conditions of a disease, disorder, or conditions described herein (e.g., a condition requiring bone tissue regeneration or bone tissue formation). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, includes compounds which are rapidly transformed in vivo to the parent compound of the above formula. Prodrugs also encompass bioequivalent compounds that, when administered to a human, lead to the in vivo formation of therapeutic. Preferably, prodrugs of the compounds of the present invention are pharmaceutically acceptable.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” includes the amount of therapeutic present in the plasma of a treated subject (e.g., as measured in a rabbit using an assay described below or in a human).

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIGS. 1A-1E show that inhibition of notch signaling by OA enhances inducted MSCs osteogenic differentiation. (FIG. 1A) Cell viability assays showed a significant increase of cell death was only observed in high dose of OA (30 μM) treatment for 12 hours. (FIG. 1 B) Quantification of gene expression of osteogenic markers indicates a significant increase (p<0.05) of early stage markers of ALP, Runx2, and type I collagen (Col1a1); and no significant change was observed in expression of later markers Osteocalcin (OC) and Osteopontin (OPN). Data is the means ±s.d. of three independent experiments performed in duplicate and the shRNA-lentivirus control (Co) gene expression level was set at 1. (*p<0.05 compared with control) (FIG. 1C) An increase in osteogenic nodule formation was observed in OA treated MSCs at day 7, with maximal staining of OA treatment at 20 μM. Scale bars, 100 μm. (FIG. 1D) Luciferase assays showed a significant decrease of Notch responsive reporter activity in OA treated MSCs in a dose dependent manner. Data is the means ±s.d. of three independent experiments performed in duplicate and all the results were normalized to internal control (*p<0.05 compared with control MSCs (Co) without OA treatment). (FIG. 1E) Real time PCR data showed a significant decrease of Notch target gene Hes1 expression at day 7 following OA treatment compared to DMSO treated control MSCs (Co).

FIGS. 2A-2E show Notch signaling blocks OA-induced osteogenesis in MSC culture. NICD1 lentivirus infected MSCs were treated with OA before being harvested for western blot, ALP staining, and RT-PCR analysis. (FIG. 2A) Western Blot showed a significant increase of NICD protein expression at day 3 following NICD1 lentiviral infection. Full-length blots/gels are shown in FIG. 6. (FIG. 2B) Overexpression of NICD1 resulted in decreased ALP staining at day 7, compared to GFP-lentirvirus infected controls (Co). (Magnification: ×5). (FIGS. 2C, 2D, and 2E). Real time PCR data showed a significant increase of ALP, Runx2, Col1a1 expression at day 7 following OA treatment and these induced expression was significantly reduced by NICD1 lentiviral infection compared to GFP-lentirvirus infected controls (Co). Data is the means ±s.d. of three independent experiments performed in duplicate and the control gene expression level at day 7 was set at 1 (*p<0.05 compared with control).

FIGS. 3A-3D show the synergistic effect of OA and BMP2 on MSC osteogenic differentiation. (FIG. 3A) ALP staining showed a significant increase of ALP activity in MSCs treated with either OA or BMP2, and this increase is further enhanced by the combined treatment at day 7. (FIG. 3B) Real time RT-PCR analysis reveals that expression of both early and later osteogenic markers (ALP, Runx2, OC, OPN) were significantly enhanced by combined treatment with OA and BMP2 at day 7 when compared with either OA or BMP2 alone treated MSCs. PCR data is means ±s.d. of three independent experiments performed in duplicate and all the results were normalized to control (*p<0.05 compared with control MSCs (Co) without treatment). (FIG. 3C) RT-PCR data shows treatment of OA in MSCs did not cause a significant change in gene expression of phosphor-smad1/5/8 at day 7. Data is the means ±s.d. of three independent experiments. (*p<0.05 compared with control MSCs). (FIG. 3D) Western Blot shows phosphor-smad1/5/8 (p-smad1/5/8) protein levels were not affected by OA treatment in MSC culture at day 7. β-actin was used as a loading control. Full-length blots/gels are presented in FIG. 6.

FIGS. 4A and 4B show OA treatment enhances MSC ectopic bone formation in vivo. (FIG. 4A) Top panel: Gross observation of transplanted MSC/HA pellet immediately after fixation. Middle panel: Masson's Trichrome staining of tissue sections from the ectopic ossicle formation samples at 6 weeks after implanted with MSCs that were previously mixed with OA and/or BMP2. Blue staining denotes a Col1a1 rich bone-like matrix in the ossicles. Low panel: Immunohistochemistry staining of osteopontin in transplanted MSC/HA pellet. (FIG. 4B) Areas of bone-like matrix (blue) staining on Masson's Trichrome stained sections were quantified vs total area using ImageJ for all groups (n=3; magnification, ×20).

FIG. 5 shows a possible model of OA in regulation of MSC osteogenic differentiation. Inhibition of Notch signaling by OA in MSC culture stimulates osteogenic gene expression (ALP, Runx2, Collagen I) that may lead to enhanced MSC osteogenic differentiation in the early stage of osteoblast formation. OA works parallel with BMP signaling to accelerate MSC differentiation toward to osteoblasts.

FIGS. 6A-6D show uncropped images for Western blots in this disclosure. (FIG. 6A)Full-length blots/gels of NICD1 in FIG. 2A. (FIG. 6B) Full-length blots/gels of β-actin in FIG. 2A. (FIG. 6C) Full-length blots/gels of p-Smad1/5/8 in FIG. 3D. (FIG. 6D) Full-length blots/gels of β-actin in FIG. 3D.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

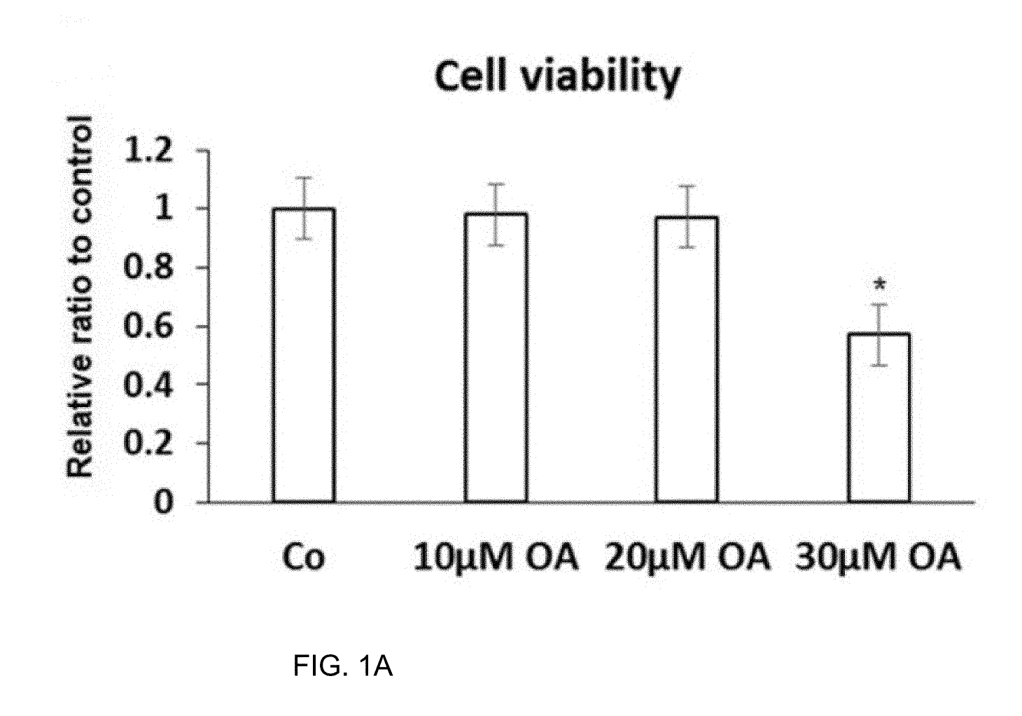

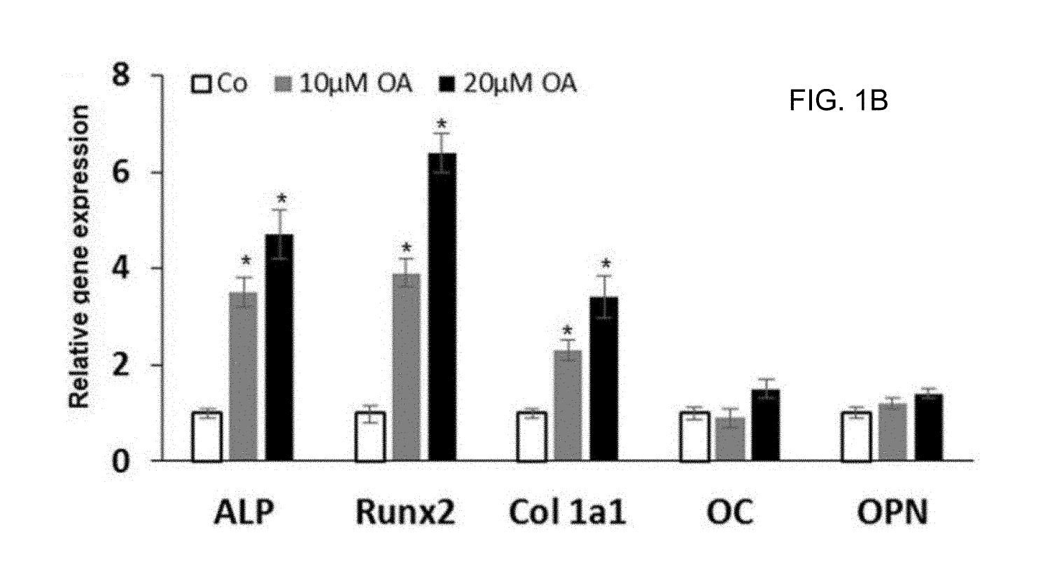

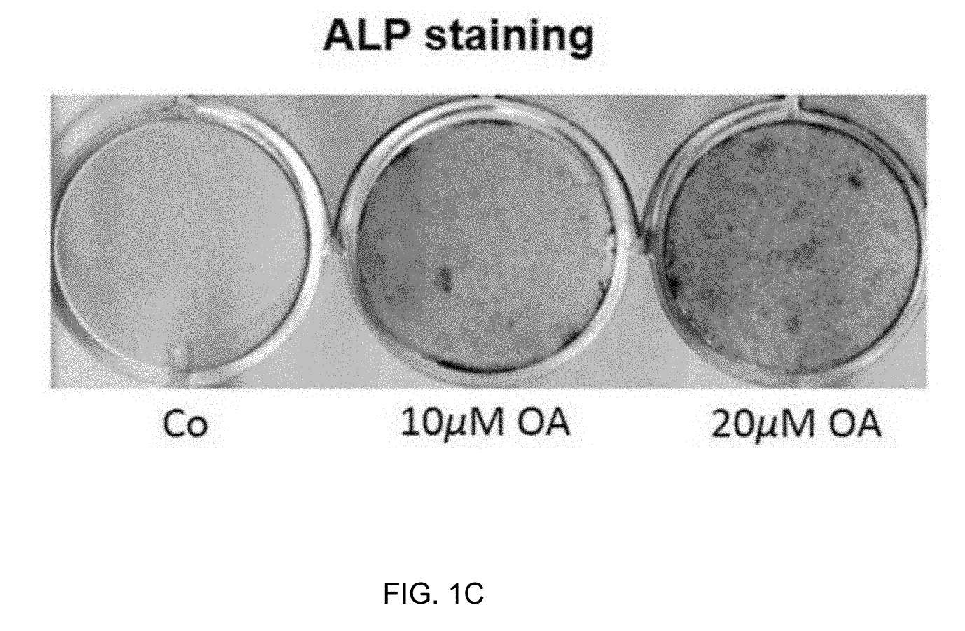

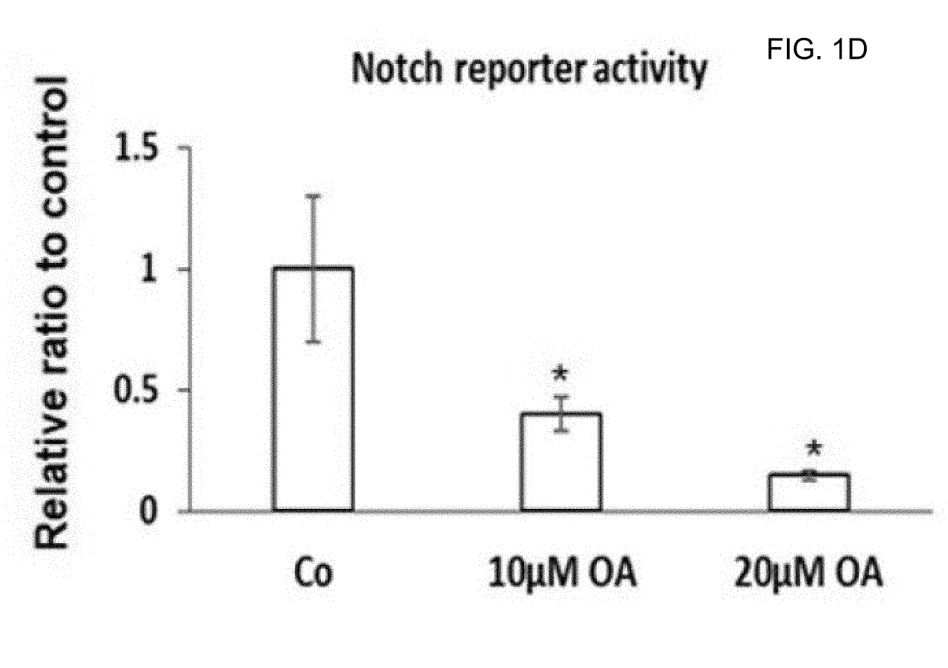

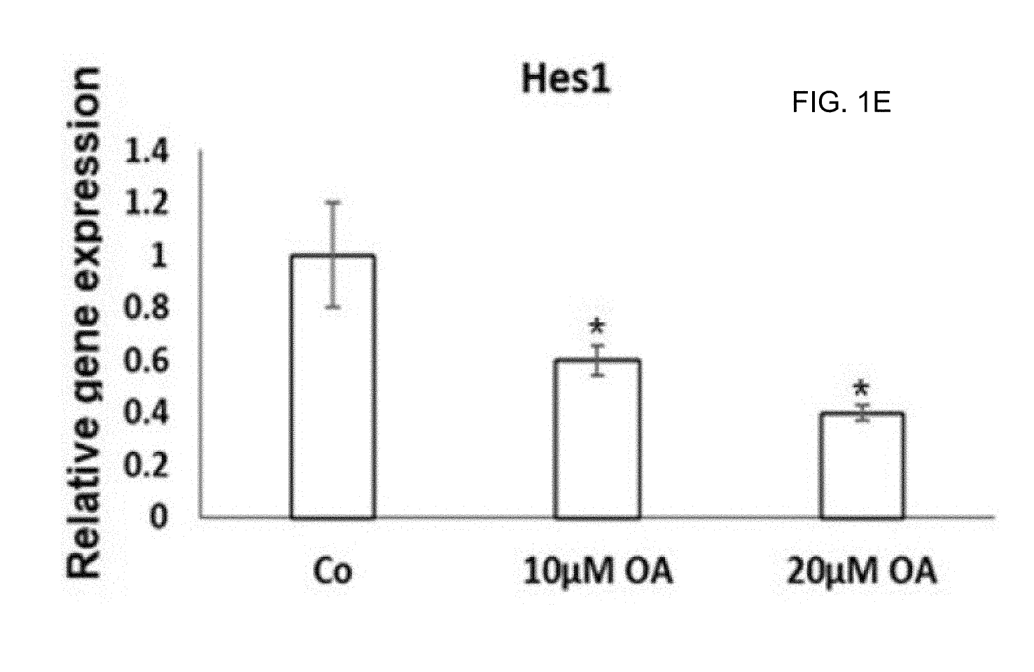

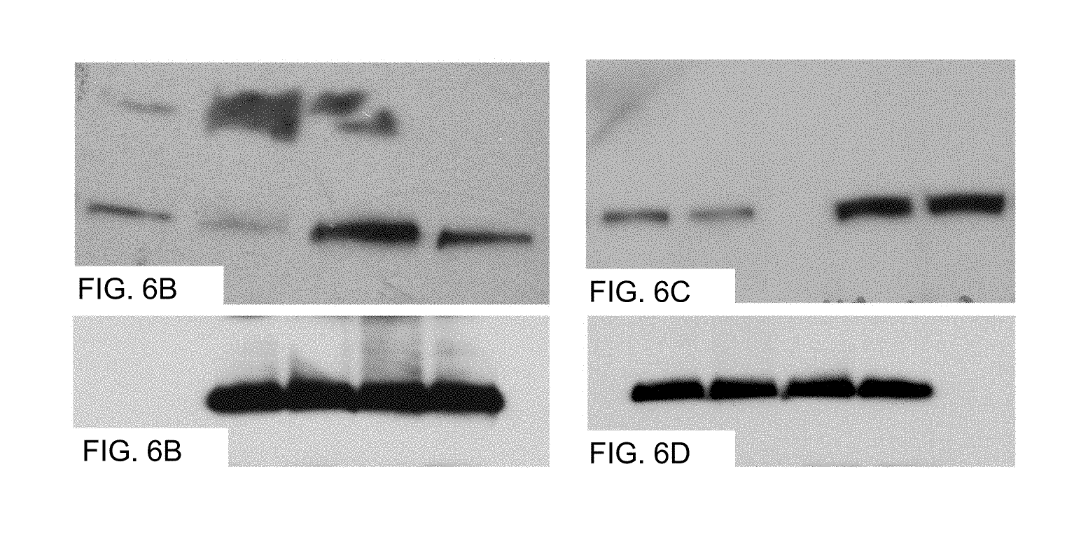

Turning now to FIGS. 1A to 6D, a brief description concerning the various components of the present invention will now be briefly discussed.

Oleanolic acid (OA), a pentacyclic triterpenoid, modulates multiple signaling pathways in a variety of cell linages. But the mechanisms underlying OA-mediated mesenchymal stromal cell (MSC) osteogenic differentiation are not known. In a study, the inventor examined effects of OA on cell viability, osteogenic differentiation in MSCs, and the involvement of Notch and BMP signaling. OA induced bone marrow derived MSC differentiation towards osteoprogenitor cells and inhibited Notch signaling in a dose dependent manner. Constitutive activation of Notch signaling fully blocked OA induced MSC osteogenic differentiation. The expression level of early osteogenic marker genes, ALP, Runx2, and type I collagen, which play a critical role in MSC to osteoblast transition and serves as a downstream target of BMP signaling, was significantly induced by OA. Furthermore, BMP2 mediated MSC osteogenic differentiation was significantly enhance by OA treatment, indicating a synergistic effect between BMP2 and OA. The inventor's results suggest that OA is a promising bioactive agent for bone tissue regeneration, and inhibition of Notch signaling may be required for its osteogenic effects on MSCs.

As a natural pentacyclic triterpenoid, oleanolic acid [(3β)-3-hydroxyolean-12-en-28-oic acid] (OA) is an aglycone of many saponins. OA exists widely in food products (vegetable oils) and could be extracted from the leaves and roots of many plant species, such as Olea europaea, Viscum album L., and Aralia chinensis I. OA exhibits various biological properties, including anti-inflammatory, antidiabetic, and hepatoprotective effects. After absorption, OA is mainly distributed and transformed in the liver. OA protects mice from various hepatotoxicants, such as carbon tetrachloride, acetaminophen, bromobenzene, and thioacetamide, and OA displayed no significant cytotoxicity to normal cells. OA exhibits metabolic activity in many cellular processes, including apoptosis, cell cycle arrest, and differentiation. The effects of OA on stem cell osteogenic differentiation have not been investigated in detail, and the underlying mechanisms responsible for bone forming effect of OA are still unknown. Various signaling factors have been implicated in the regulation of MSC osteoblastic differentiation. The inventor has identified the Notch pathway as an important inducer of MSC proliferation, and as an inhibitor of MSC differentiation during mouse limb-bud and postnatal bone development. Activation of Notch signaling induces cleavage and release of the Notch intracellular domain (NICD), which translocates from the cell surface into the nucleus to activate target gene expression of Hes1 via a NICD-RBPJK-MAML transcriptional complex.

In the disclosed study, the inventor first studied the effect of OA on Notch signaling-mediated MSC osteogenic differentiation, and then determined the effect of OA on BMP-induced osteogenesis.

Materials and Methods

Cell culture and treatment: The human MSCs were purchased from Lonza. Oleanolic acid was obtained from Sigma-Aldrich (Aldrich-5504). A 100 mM solution of OA was prepared in dimethyl sulfoxide (DMSO), and all test concentrations were prepared by diluting the stock solution in cell culture medium. Recombinant human BMP2 was purchased from R&D. The cells were cultured in DMEM supplemented with 10% fetal bovine serum (Gibco BRL, Rockville, Md.). After expansion, cells were exposed to media containing OA and/or BMP2 for certain days. Since the half-life of OA is around eight to 16 hours, the inventor decided to change media once a day for up to seven days to induce MSC differentiation. At the end, cells were harvested at different time points for analyses as described below.

Luciferase assay: 80% confluence MSCs were transfected with Notch responsive RBPJ-Luc and SV40-Renilla-Luc (Promega) in the presence of Lipofectamine 2000 (Invitrogen) followed by OA treatment. At 24 hours after transfection and treatment, lysates were analyzed with a Dual Luciferase Assay Kit (Promega).

Cell viability assay: MSC viability was measured using MTT (3-(4, 5-dimethylthiazole-2-yl)-2, 5-diphenyl tetrazolium bromide) based in vitro toxicology assay kit (Sigma, St. Louis, Mo., USA). Briefly, cells were seeded in 96-well plates. After growing into 50-60% confluence, cells were exposed to various concentrations of OA for 12 hours, then the supernatant was discarded, and cells were treated with 0.5 mg/ml MTT staining solution for another four hours. After incubation, the supernatant was removed and 50 μl of solubilization buffer provided by the Sigma kit with 0.5% DMSO was added. DMSO was added to ensure total solubility of the formazan crystals. Plates were shaken for two minutes, and the absorbance recorded at 590 nm on an automated microtiter plate reader. The percent viability was expressed as a percentage of that in the vehicle control (with subtraction of background absorbance).

MSC osteogenic differentiation assay: Osteogenic differentiation assays of MSCs were performed using the Osteogenic differentiation bulletKit® (Lonza). MSCs were cultured at 200,000 cells/well in 6-well plates to confluence. Stem cell growth media was then replaced with osteogenic media supplied with or without OA and BMP2. After seven days of culture, alkaline phosphatase (ALP) staining, RNA, and protein isolation were performed as described in our published protocols.

Real time RT-PCR: cDNA was synthesized from 1 μg total RNA using the SuperScript III reverse transcriptase kit (Invitrogen) in a final volume of 20 μl. Primers were designed with the IDT SCI primer design tool (Integrated DNA Technologies, San Diego, Calif.). RT-PCR experiments were performed with a Bio-Rad C1000 thermal cycler (Bio-Rad, Hercules, Calif.), and real-time PCR experiments were performed with an ABI prism 7500 (Applied Biosystems, Grand Island, N.Y.) in triplicate. Sequence for each primer pair were: Hes1, forward primer 5′-TTCCTCCTCCCCGGTGGCTG-3′, reverse primer 5′-TGCCCTTCGCCTCTTCTCCA-3′; ALP, forward primer 5′-GGGCATTGTGACTACCACTC-3′, reverse primer 5′-AGTCAGGTTGTT CCGATTCA-3′; Runx2, forward primer 5′-CACTGCCACCTCTGACTTCT-3′, reverse primer 5′-CACCATCATTCTGGTTAGGC-3′; Osteocalcin (OC), forward primer 5′-TGGCCATGCTGACTGCAGCC-3′, reverse primer 5′-TGGGTAGGCGTCCCCCATGG-3′; Osteopontin (OPN), forward primer 5′-AAGGAACCAAAGCATCAAGAATTAG-3′, reverse primer 5′-AGATGTCATCAGGCAGCTTGAC-3′; Type I collagen (Col1a1), forward primer 5′-GTTTGGCCTGAAGCAGAGAC-3′, reverse primer 5′-TCTAAATGGGCCACTTCCAC-3′; β-actin, forward primer 5′-ACCACAGTCCATGCCATCAC-3′; reverse primer 5′-TCCACCACCC TGTTGCTGTA-3′. Samples were analyzed in triplicate, and the raw data consisted of PCR cycle numbers required to reach a fluorescence threshold level. The relative expression level of target genes was normalized to (β-actin gene.

Lentivirus-mediated Notch activation: cDNAs encoding human Notch1-NICD was cloned into the EF.v-CMV lentiviral vector and sequence verified. Lentivirus production and concentration was performed as previously described. Briefly, VSV.G-pseudotyped recombinant lentiviruses were produced by transient transfection of the transducing vector into 293 T cells, along with two packaging vectors: pMD.G, a VSV.G envelope-expressing plasmid, and pCMVΔR8.91 (Invitrogen, Carlsbad, Calif., USA), containing the HIV-1 gag/pol, tat, and rev genes (1.5 μg: 2.0 μg: 0.5 μg ratio of these three vectors). Viral supernatants were collected at 24, 48 and 72 hours after transfection, and concentrated using filtration columns (Centricon Plus-20, molecular weight cutoff =100 kDa; Millipore, Bedford, Mass., USA). For lentiviral infection, 10 ml high-density (10,000 cell/ml) MSCs were seeded in 6-well plates and incubated for two hours at 37° C. prior to being added with osteogenic medium supplied with NICD1 lentivirus, and 8 μg/ml polybrene. GFP-lentivirus was used in this experiment as a control. The infected MSC cultures were harvested at day seven for ALP staining, protein and RNA isolation.

Western blot analysis: MSCs cultured with or without OA and BMP2 treatment were lysed in RIPA buffer (10 mM Tris-HCI, 1 mM EDTA, 1 sodium dodecyl sulfate [SDS], 1% NP-40, 1:100 proteinase inhibitor cocktail, 50 mM β-glycerophosphate, 50 mM sodium fluoride). The samples were separated on a 10% SDS polyacrylamide gel and transferred to polyvinylidene difluoride (PVDF) membranes with a semi-dry transfer apparatus (Bio-Rad). The membranes were blotted with 5% dehydrated milk for two hours, and then, incubated with primary antibodies overnight. The immune complexes were incubated with horseradish peroxidase-conjugated anti-rabbit or anti-mouse IgG (Promega, USA), and visualized with SuperSignal reagents (Pierce, USA). Primary polyclonal antibodies against NICD1 and phosphor-smad1/5/8 (Cell Signaling, USA) were used. A primary monoclonal antibody was used to detect the housekeeping protein, β-actin (Sigma-Aldrich, USA).

In vivo ectopic bone-formation assay: All animal studies were reviewed and approved by the Animal Ethics Committee of Louisiana State University Health Sciences Center. 1.0×10⁶ total MSCs were re-suspended in 80 μl of MSC growth media containing 20 μM OA and gently mixed with 50 mg of hydroxyapatite (HA) powder (Himed). The composites were then centrifuged for two minutes at 200 rpm to form a MSC/HA pellet. For ectopic bone formation surgery, ten 8-week-old NOD.CB17^{-Prkdescid/J} mice (Charles River) (up to 2 implants for each animal) were anesthetized and the dorsal skin was cleaned with 70% ethanol. Two incisions of ˜1 cm in length were performed on the opposite flanks of the back, and then a pocket was formed by blunt dissection. Finally the MSC/HA pellets were implanted. Six weeks post-surgery, transplanted BMSC/HA pellets were harvested for gross observation and Masson's trichrome staining. Immunohistochemistry (IHC) staining was performed using anti-osteopontin antibody (7C5H12, Abcam, USA) according to our published protocols. All animal work was performed in accordance with institution approved guidelines.

Statistical Analysis: All experiments were repeated at least three times independently. Data was presented as mean ±s.d. Statistical significance among the groups was assessed with one-way ANOVA. The level of significance was p<0.05.

Results

OA induces osteogenic differentiation of MSCs while inhibiting Notch signaling in a dose-dependent manner. In order for bones to regenerate, specific mesenchymal stem cells (MSCs) surrounding the injured site have to first differentiate into osteoprogenitor cells, and then become functional osteoblasts. To examine the effects of OA on the cell viability in MSCs, we performed the in vitro cell viability assays, after 12 hours treatment of OA in three different doses, a significant cell death was observed in cells with the dose of 30 μM, and no cell death was noticed in cultures with low doses of 10 μM and 20 μM treatment (FIG. 1A). Therefore, 10 μM and 20 μM of OA was used in subsequent experiment. MSC osteogenic differentiation was further observed in osteogenic differentiation cultures following treatment with two different doses of OA for seven days. Cultures from OA treated MSCs exhibited enhanced expression of early osteogenic markers, Col1a1, ALP, and Runx2, as compared to cultures from control MSCs treated with DMSO (FIG. 1B). Interestingly, the later osteogenic markers OC and OPN remained unchanged (FIG. 1B), indicating OA mainly functions at the onset stage of MSC osteogenic differentiation. Finally, ALP staining in cultures indicated a dose dependent increase of MSC osteogenic differentiation in OA treated MSCs by showing a stronger staining (FIG. 1C) further confirmed this enhanced osteogenic differentiation in OA treated MSCs.

Accumulated evidence indicates that Notch signaling plays a key role in regulating cell growth and differentiation during development. The inventor has identified the RBPjk-dependent Notch pathway as an important inducer of MSC proliferation, and an inhibitor of MSC differentiation during mouse limb-bud and postnatal bone development. In this study, the inventor further tested whether Notch signaling is also involved in OA-mediated osteogenesis. To do so, MSCs were first transfected with the RBPjk-dependent NOTCH-responsive luciferase reporter, and then cultured in medium containing 0, 10 μM and 20 μM OA for 48 hours. The results revealed that OA inhibited Notch-responsive luciferase activity dose dependently in MSCs (FIG. 1D). Moreover, down-regulation of Notch target gene Hes1 expression was also observed in OA treated MSCs (FIG. 1E), which further confirmed the inhibitory effect of OA on Notch signaling. These results suggested that OA has the ability to promote osteogenesis while preserving cell viability at the concentration of 20 μM, so 20 μM OA was used for treatment for subsequent experiments.

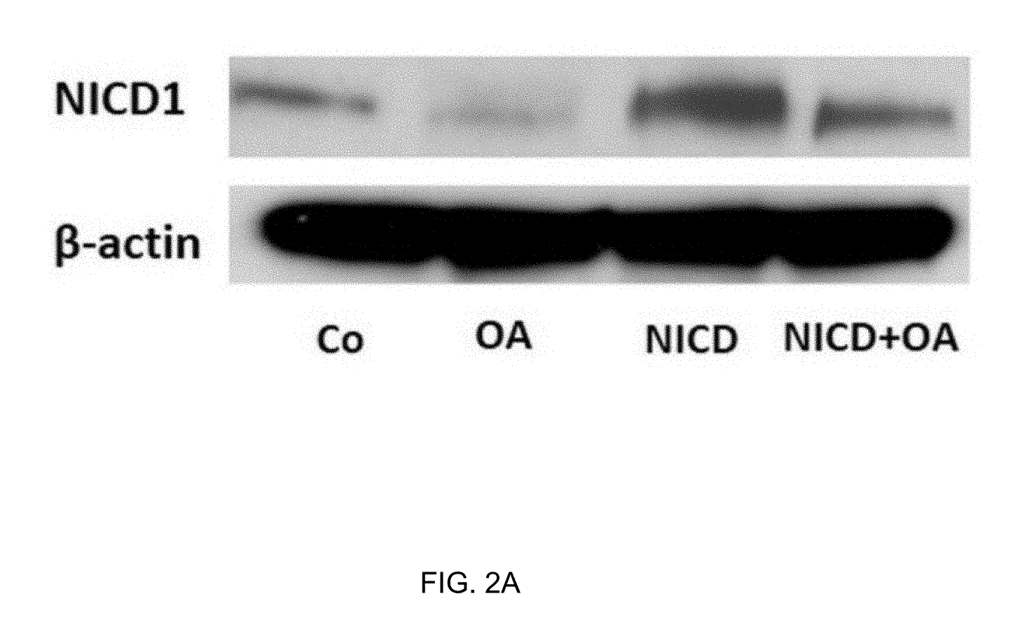

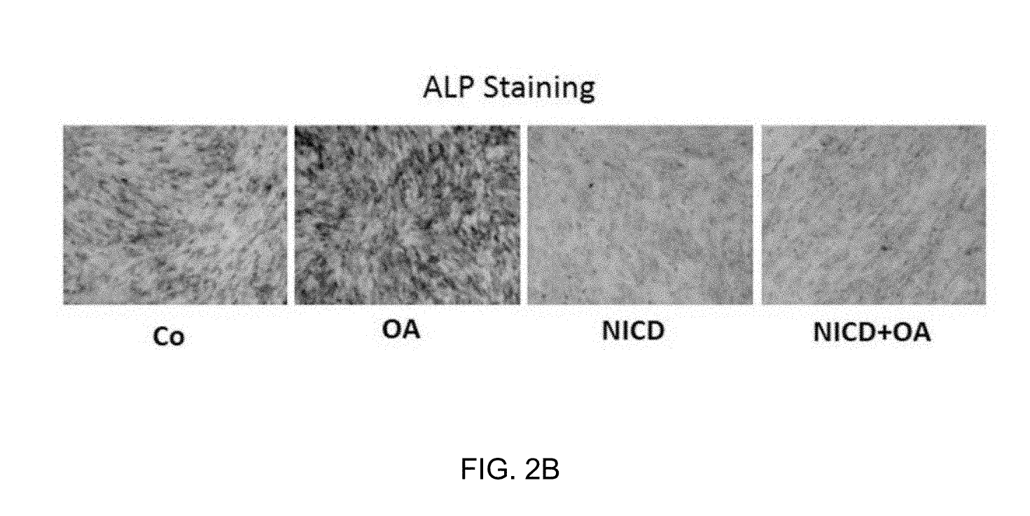

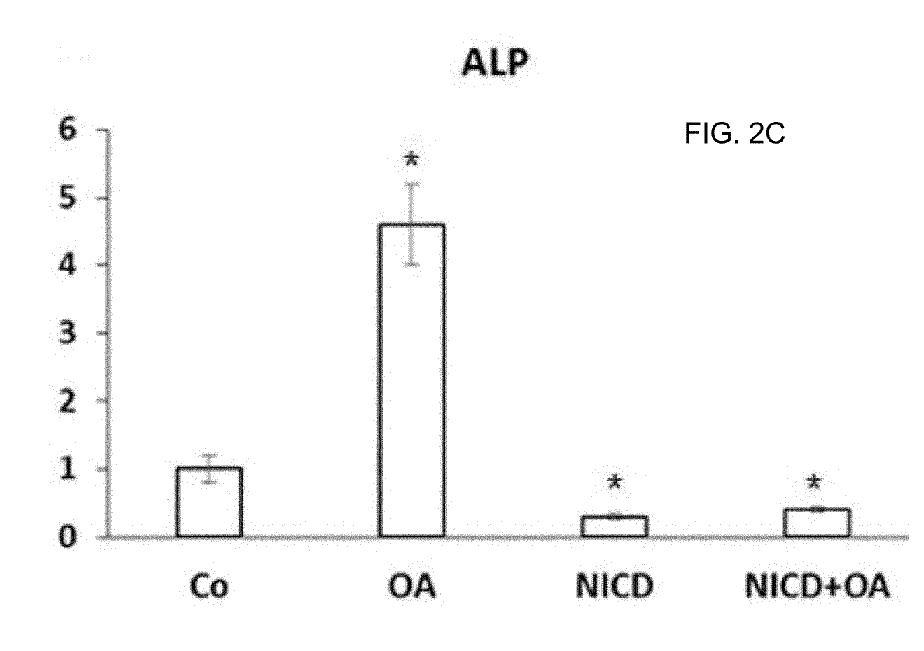

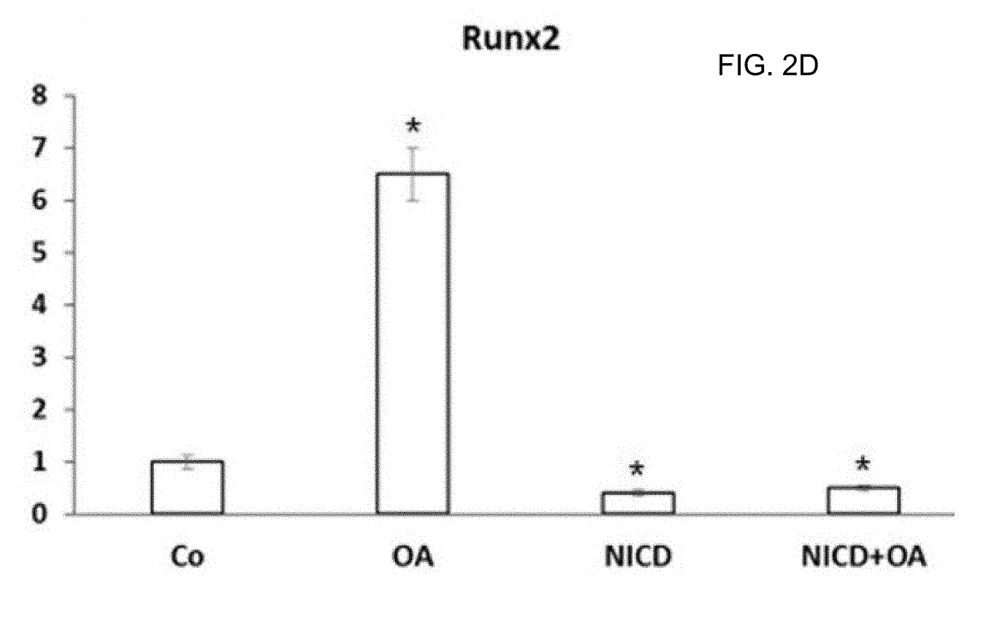

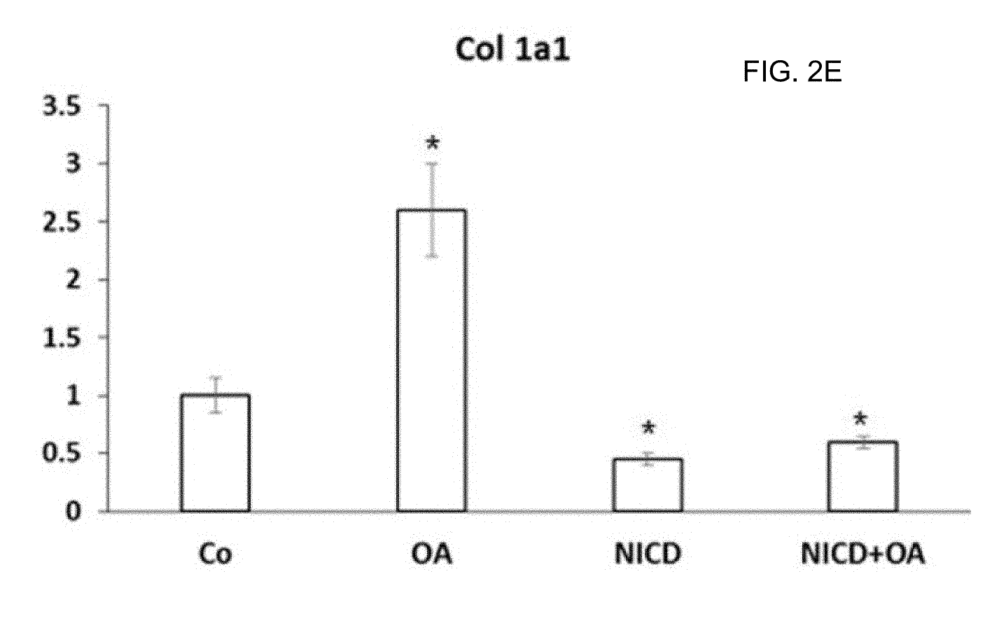

Constitutive Notch activation blocks OA-induced osteogenic differentiation: Although a reduction of Notch signaling activity was observed in OA treated MSCs, whether Notch inhibition is specifically required for OA mediated osteogenesis was unknown. To determine whether Notch signaling is a key player responsible for OA effect on MSC osteogenesis, Notch signaling was activated in MSCs by overexpressing Notch intracellular domain (NICD1) with lentivirus transduction. The western blot data in FIG. 2A showed that the expression of NICD was significantly reduced by OA treatment, and constitutive overexpression of NICD1 by lentivirus significantly induced Notch signaling in both OA treated and untreated MSCs. Next, ALP staining was performed in NICD1 expressing MSCs. The image in FIG. 2B clearly shows a weak staining in NICD1 infected MSCs when compared to control cells. More importantly, OA-induced ALP staining was totally blocked by overexpression of NICD1 suggesting that down regulation of Notch signaling is required for OA induced MSC osteogenic differentiation. The expression of early osteogenic markers, such as ALP, Runx2 and Col1a1, was also examined by RT-PCR. As expected, the expression of these genes was significantly reduced in both Notch activated MSCs with or without treatment of OA when compared to either control or OA treated MSCs (FIGS. 2C, 2D, 2E).

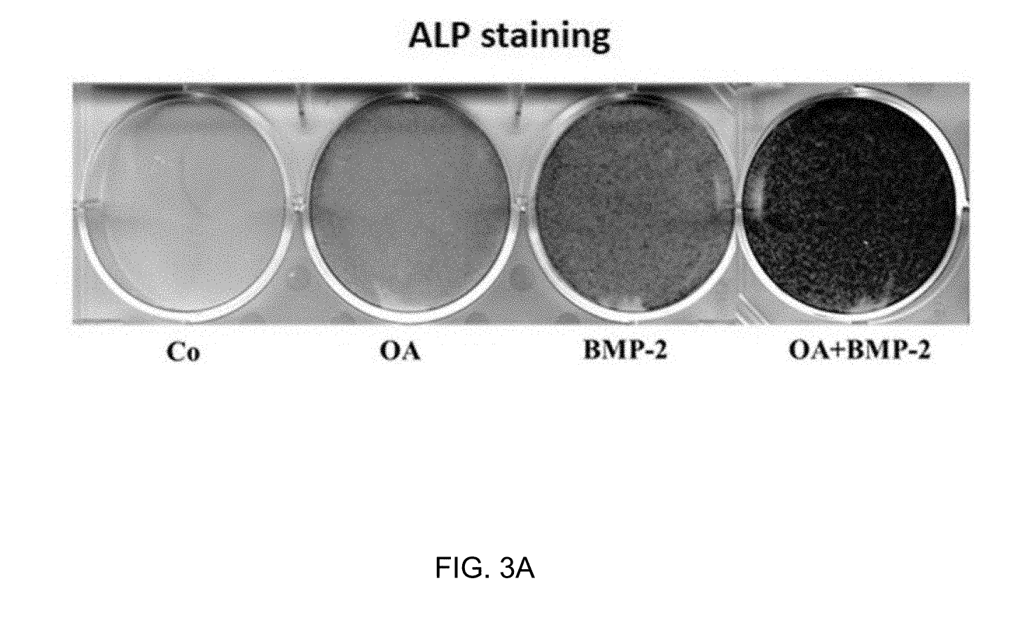

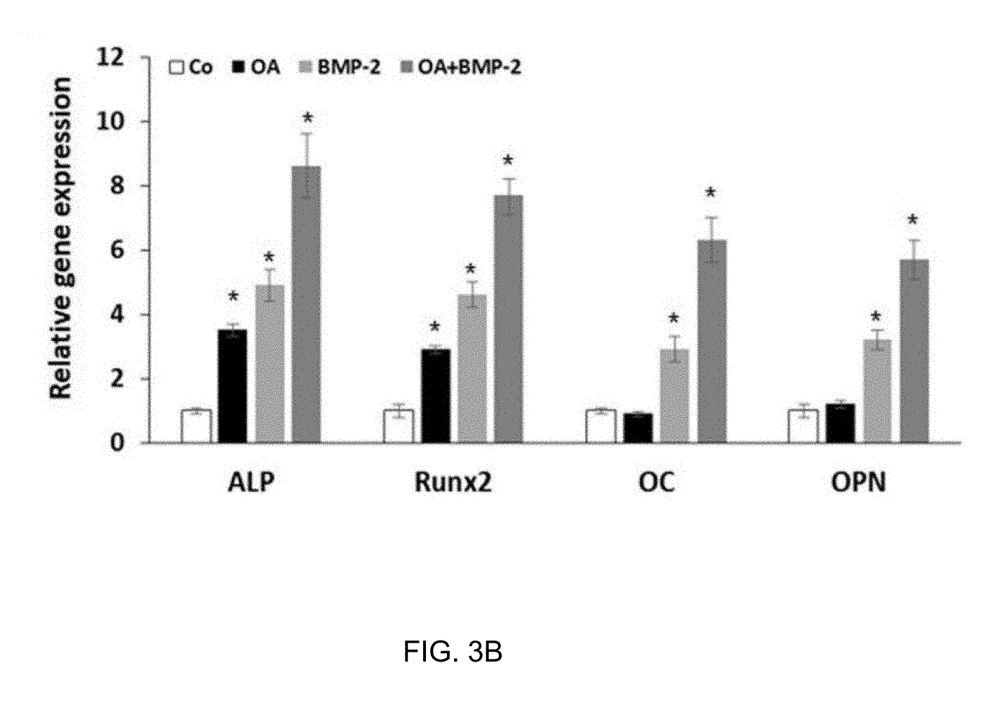

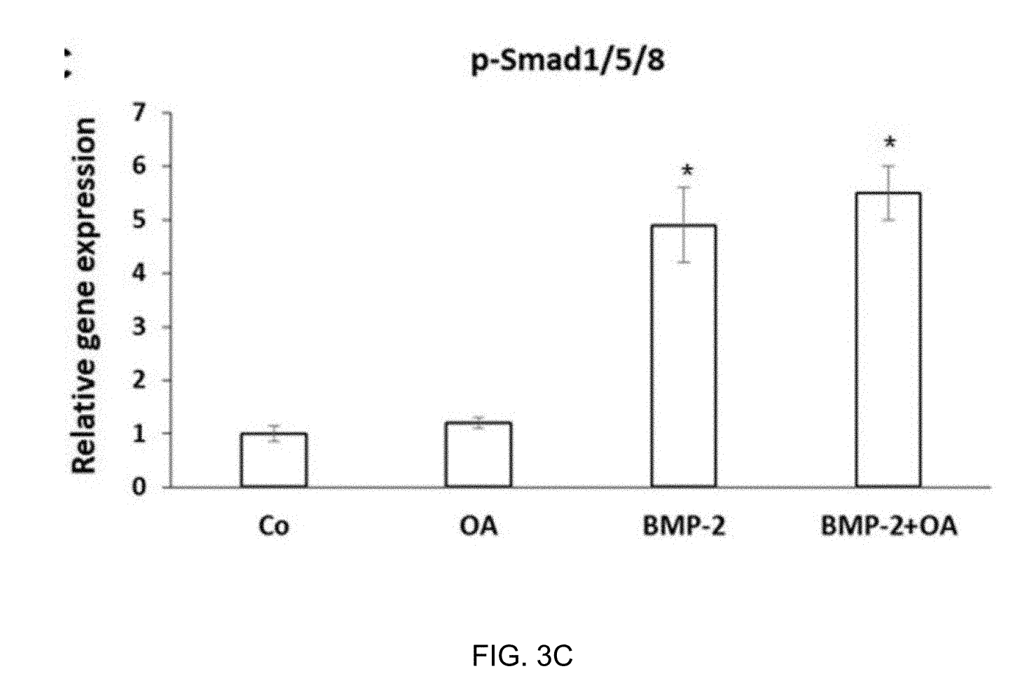

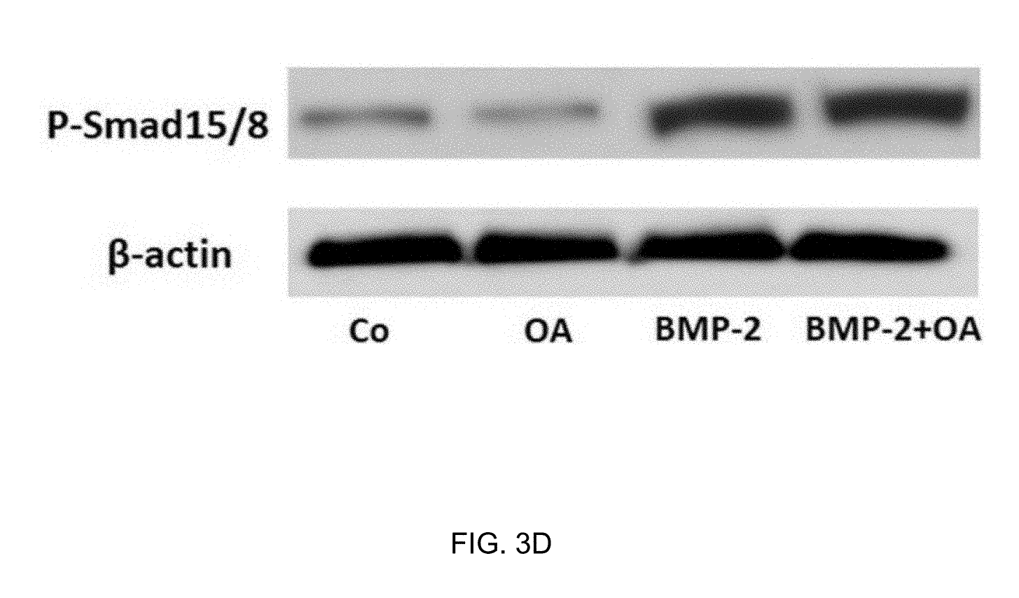

OA enhances BMP2-induced MSC osteogenic differentiation: BMP2 is well known for its osteogenic properties and is used clinically in the spine fusion surgery. Since these osteogenic markers induced by OA (FIG. 1) are also regulated by BMP signaling, we further investigated the effects of OA on BMP2 stimulated osteogenesis. To do so, MSCs were first cultured with OA (20 μM) and/or BMP2 (200 ng/ml) for seven days in osteogenic medium, and then harvested for ALP staining and total RNA isolation. ALP staining image in FIG. 3A showed that ALP activity was significantly enhanced by the combination of OA and BMP2 compared with groups treated with either OA or BMP2 alone. Real-time PCR Results further confirmed that treatment with a combination of OA and BMP2 synergistically enhanced the gene expression of osteogenic markers, ALP, Col1a1, Runx2, OC, and OPN (FIG. 2A). As phospho-smad1/5/8 (p-smad1/5/8) is the key downstream factor in BMP signaling, the expression of p-smad1/5/8 in OA-mediated MSC osteogenic differentiation was next measured. As shown in FIG. 3C, BMP2 treatment significantly increased gene expression of p-smad1/5/8 in MSCs. In contrast, OA treatment did not show any effects on the expression of p-smad1/5/8 in both MSCs with or without BMP2 treatment. To confirm these results, total protein was isolated from BMP2 and/or OA treated MSCs. Consistent with RT-PCR data, the western blot data clearly showed that no significant difference of p-smad1/5/8 expression was observed between control and OA treated MSCs; thus, suggesting OA induced MSC osteogenic differentiation in a BMP signaling independent manner.

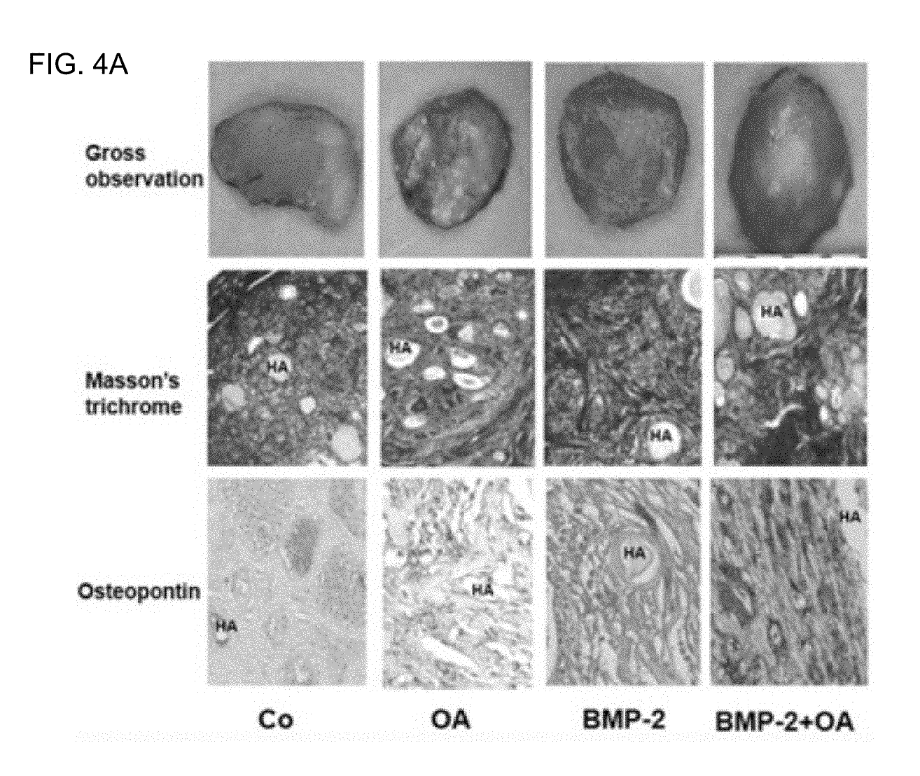

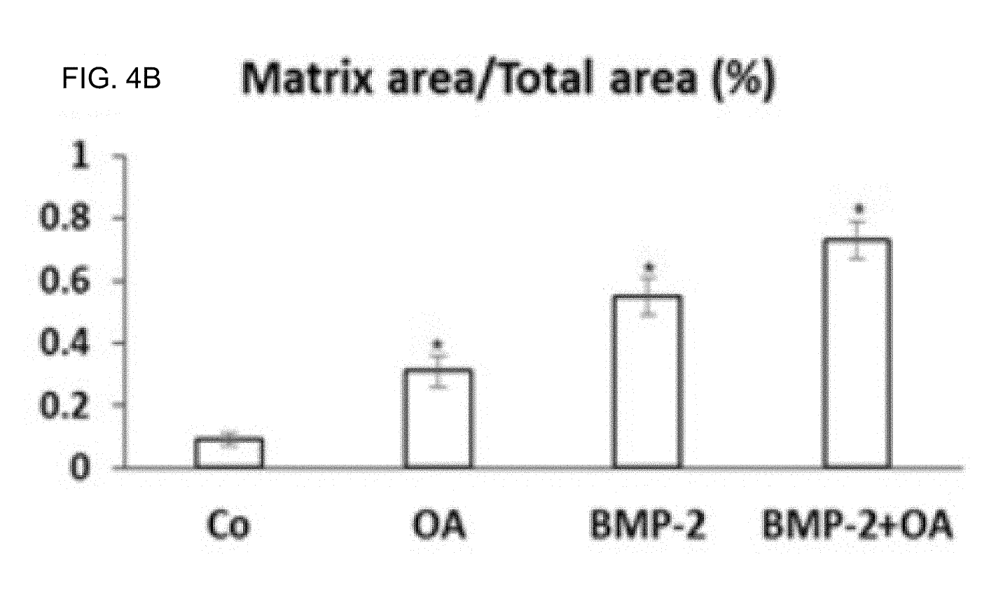

OA enhances BMP2-mediated ectopic bone formation: To further confirm the capacity of OA induced osteogenesis in vivo, ectopic ossicle assays using 8-week old nude mice was performed. MSCs were first dissolved in PBS containing 20 μM OA, then mixed with HA ceramic powder, and centrifuged into MSC/HA pellets prior to subcutaneous transplantation into nude mice. After six weeks of growth and differentiation, the pellets were harvested for gross observation, Masson's trichrome and immunohistochemistry staining (FIG. 4A). Gross observation of harvested ectopic bone formation pellet showed a round-shaped pellet with 2-3 mm in diameter from each groups. No significant difference was noticed regarding the weight, size and stiffness of these pellets in four different groups. As the inventor expected, there was an approximately 4-fold increase of type I collagen bone matrix (blue) in the pellet area from OA treated MSCs (30%) when compared to untreated MSCs with only approximately 8% of the pellet area stained blue (FIG. 4B). Notably, the increase of bone matrix in BMP2 treated MSCs was further enhanced by OA treatment indicating a synergistic effect occurs when both OA and BMP2 are used. To further confirm this enhanced bone matrix formation, the inventor performed IHC for OPN (FIG. 4A). The results clearly showed a significant increased expression of OPN in OA and BMP treated cell pellets suggesting more osteoblastic cells in this group.

Discussion: Fractures and bone defects are common in both military and civilian populations with over 2.2 million bone repair surgeries worldwide each year. This bone repair process is regulated by multiple molecular cascades involving type I collagen matrix production and the participation of many signaling molecules, including ALP, Runx2, OC, and OPN. Despite fractures being such a common problem, there is no approved drug that speeds up bone healing, or increases the chance of proper repair in clinic. In this regard, developing an injectable drug or certain molecules that promote fracture repair and prevent non-union is increasingly receiving attention.

Oleanolic acid, a natural triterpenoid extract from various type of plants. OA or its derivative activates the JNK pathway both in normal cells and malignant cells, as well as the suppression of mTOR pathway. As these signaling pathways play an important role in mesenchymal stem cell differentiation, the effect of OA on MSC differentiation toward osteogenic cell lineage was further evaluated. After seven days of culture in osteogenic induction medium, a significantly enhanced ALP staining in OA treated MSCs demonstrated that OA could be used to induce stem cell osteogenesis in vitro. To look for possible molecules involved in this process, several osteogenic marker genes were quantified by RT-PCR. Surprisingly, only osteogenic early stage markers (ALP, Runx2, Col1a1), not later stage markers (OC,OPN), were significantly induced by OA, indicating OA plays more important role in the onset stage of osteogenesis. Since the inventor's research group was the first to demonstrate inhibition of Notch signaling promotes onset differentiation of mesenchymal stem cell toward osteogenic cell lineage, the inventor next examined the effect of OA on Notch signaling in MSCs. As expected, during OA induced MSC osteogenic differentiation, Notch signaling was significantly inhibited in a dose-dependent manner suggesting a possible regulation of Notch signaling by OA in MSCs. To further understand the importance of Notch signaling in OA induced MSC osteogenic differentiation, the inventor overexpressed NICD1 to activate Notch signaling in MSCs. The results clearly show that constitutive Notch activation fully blocked OA induced MSC osteogenesis, indicating inhibition of Notch signaling may be required for OA induced MSC osteogenic differentiation.

To further investigate whether OA induced osteogenic differentiation is close to or comparable to BMP2 induced osteogenic differentiation, ALP staining and RT-PCR was performed in MSCs treated with either OA or BMP2, or both. Although the results showed a relative weak ALP staining in OA treated MSCs when compared to BMP2 treated MSCs, OA indeed greatly enhanced the BMP2 induced osteogenic differentiation in these cells. In order to further understand the possible molecular mechanism involved in OA potentiation of BMP2 action, the effect of OA on phosphorylation of Smad1/5/8, a downstream target of BMPs that have been shown to directly stimulate bone formation and osteoblast differentiation, was investigated. OA treatment did not appear to have a significant effect on the phosphorylation of Smad1/5/8, suggesting BMP signaling is not regulated by OA in MSC osteogenic differentiation. Although Notch signaling has been inhibited by OA, the inventor has yet to identify the interaction between Notch signaling and BMP2-induced phosphorylation of Smad1/5/8.

The combination of OA and BMP2 showed a more enhanced MSC osteogenic differentiation compared with each one alone in vitro, so the inventor next performed ectopic bone formation analysis to validate this effect in vivo. After six weeks post-transplantation of OA treated MSCs under the dorsal skin, a significantly improved osteogenesis in MSCs treated with OA and BMP2 was observed when compared with each treatment alone. These consistent results from in vitro and in vitro further confirmed that OA has the ability to enhance osteogenesis and could also be used to promote BMP2 effect on bone tissue formation.

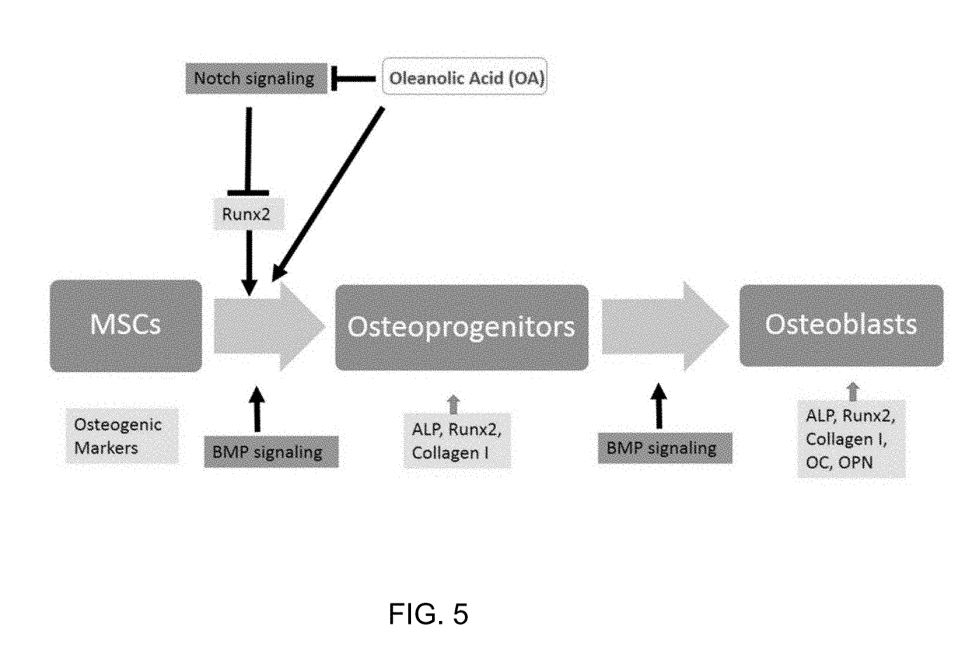

Conclusion: In summary, this study identified a natural extract from plants as a possible BMP2 substitute to promote bone tissue formation. Based on the disclosed results, it is believed that OA can induce the onset of MSC osteogenic differentiation by inhibition of Notch signaling that leads to enhanced osteogenic gene expression (ALP, Runx2, Collagen I) and subsequent bone formation, which works parallel with BMP signaling to accelerate MSC differentiation toward to osteoblasts (FIG. 5). Based on the disclosed results, it is evidenced that delivery of OA alone or in combination with BMP2 in different stages of fracture healing will lead to a better bone formation with an improved clinical safety profile.

Pharmaceutical Compositions

The methods described herein can also include the administrations of pharmaceutically acceptable compositions that include the therapeutic, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The methods described herein can include the administration of a therapeutic, or prodrugs or pharmaceutical compositions thereof, or other therapeutic agents. Exemplary therapeutics include those that enhance MCS osteogenic differentiation (including OA alone or with BMP2) and increase osteogenesis.

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palm itostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings

The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

Parenteral Administration

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery

Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992).

Oral transmucosal absorption is generally rapid because of the rich vascular supply to the mucosa and allows for a rapid rise in blood concentrations of the therapeutic.

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary formulations include rapidly disintegrating tablets and liquid-filled soft gelatin capsules.

Dosing Regimes

The present methods for treating condition requiring bone tissue regeneration or bone tissue formations are carried out by administering a therapeutic for a time and in an amount sufficient to result in decreased Notch signaling or increased osteogenic differentiation or osteogenesis.

The amount and frequency of administration of the compositions can vary depending on, for example, what is being administered, the state of the patient, and the manner of administration. In therapeutic applications, compositions can be administered to a patient suffering from condition requiring bone tissue regeneration or bone tissue formation in an amount sufficient to relieve or least partially relieve the symptoms of the condition requiring bone tissue regeneration or bone tissue formation and its complications. The dosage is likely to depend on such variables as the type and extent of progression of the condition requiring bone tissue regeneration or bone tissue formation, the severity of the condition requiring bone tissue regeneration or bone tissue formation, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of the condition requiring bone tissue regeneration or bone tissue formation or slowing its progression.

The amount of therapeutic per dose can vary. For example, a subject can receive from about 0.1 μg/kg to about 10,000 μg/kg. Generally, the therapeutic is administered in an amount such that the peak plasma concentration ranges from 150 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1°, or 2 mg/kg. In another embodiment, the administered dosage can range from 0.05-5 mmol of therapeutic (e.g., 0.089-3.9 mmol) or 0.1-50 μl of therapeutic (e.g., 0.1-25 μmol or 0.4-20 μmol).

The plasma concentration of therapeutic can also be measured according to methods known in the art. Exemplary peak plasma concentrations of therapeutic can range from 0.05-10 μM, 0.1-10 μM, 0.1-5.0 μM, or 0.1-1 μM. Alternatively, the average plasma levels of therapeutic can range from 400-1200 μM (e.g., between 500-1000 μM) or between 50-250 μM (e.g., between 40-200 μM). In some embodiments where sustained release of the drug is desirable, the peak plasma concentrations (e.g., of therapeutic) may be maintained for 6-14 hours, e.g., for 6-12 or 6-10 hours. In other embodiments where immediate release of the drug is desirable, the peak plasma concentration (e.g., of therapeutic) may be maintained for, e.g., 30 minutes.

The frequency of treatment may also vary. The subject can be treated one or more times per day with therapeutic (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

Kits

Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce symptoms and/or underlying cause of the condition requiring bone tissue regeneration or bone tissue formation.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A method of increasing mesenchymal stromal cell osteogenic potential in a patient comprising:
administering to the patient a therapeutic that inhibits Notch signaling.

2. The method of claim 1 wherein the therapeutic includes oleanolic acid or a pharmaceutically acceptable salt, solvate, ester, amide, clathrate, stereoisomer, enantiomer, prodrug or analog thereof.

3. The method of claim 2 wherein the therapeutic further includes growth factor bone morphogenetic protein 2 (BMP2) or a pharmaceutically acceptable salt, solvate, ester, amide, clathrate, stereoisomer, enantiomer, prodrug or analog thereof.

4. A method of stimulating one of bone tissue regeneration and bone tissue formation in a mammal comprising:
administering to the mammal a first therapeutic containing Oleanolic acid.

5. The method of claim 1 further comprising administering a second therapeutic for one of bone tissue regeneration and bone tissue formation which is distinct from the first therapeutic.

6. The method of claim 5 wherein the second therapeutic does not contain Oleanoic acid or a pharmaceutically acceptable salt, solvate, ester, amide, clathrate, stereoisomer, enantiomer, prodrug or analog thereof.

7. The method of claim 5 wherein the second therapeutic contains growth factor bone morphogenetic protein 2 (BMP2) or a pharmaceutically acceptable salt, solvate, ester, amide, clathrate, stereoisomer, enantiomer, prodrug or analog thereof.

8. The method of claim 4 wherein the first therapeutic is injected into the mammal.

9. The method of claim 4 wherein the one of bone tissue regeneration and bone tissue formation is ectopic bone formation.

10. A pharmaceutical product comprising:
a first therapeutic containing Oleanolic acid or a pharmaceutically acceptable salt, solvate, ester, amide, clathrate, stereoisomer, enantiomer, prodrug or analog thereof.

11. The pharmaceutical product containing a second therapeutic distinct from the first therapeutic.

12. The pharmaceutical product of claim 11 wherein the second therapeutic contains growth factor bone morphogenetic protein 2 (BMP2) or a pharmaceutically acceptable salt, solvate, ester, amide, clathrate, stereoisomer, enantiomer, prodrug or analog thereof.


SEQUENCE LISTING (14 sequences)

SEQ ID NO: 1 - DNA, 20 bp, Homo sapiens
ttcctcctccccggtggctg

SEQ ID NO: 2 - DNA, 20 bp, Homo sapiens
tgcccttcgcctcttctcca

SEQ ID NO: 3 - DNA, 20 bp, Homo sapiens
gggcattgtgactaccactc

SEQ ID NO: 4 - DNA, 20 bp, Homo sapiens
agtcaggttgttccgattca

SEQ ID NO: 5 - DNA, 20 bp, Homo sapiens
cactgccacctctgacttct

SEQ ID NO: 6 - DNA, 20 bp, Homo sapiens
caccatcattctggttaggc

SEQ ID NO: 7 - DNA, 20 bp, Homo sapiens
tggccatgctgactgcagcc

SEQ ID NO: 8 - DNA, 20 bp, Homo sapiens
tgggtaggcgtcccccatgg

SEQ ID NO: 9 - DNA, 25 bp, Homo sapiens
aaggaaccaaagcatcaagaattag

SEQ ID NO: 10 - DNA, 22 bp, Homo sapiens
agatgtcatcaggcagcttgac

SEQ ID NO: 11 - DNA, 20 bp, Homo sapiens
gtttggcctgaagcagagac

SEQ ID NO: 12 - DNA, 20 bp, Homo sapiens
tctaaatgggccacttccac

SEQ ID NO: 13 - DNA, 20 bp, Homo sapiens
tccaccaccctgttgctgta

SEQ ID NO: 14 - DNA, 20 bp, Homo sapiens
accacagtccatgccatcac